(19) United States
(12) Patent Application Publication
(10) Pub. No.: US 2026/0014368 A1
(43) Pub. Date: Jan. 15, 2026

(54) ELECTRODE STRUCTURE FOR SKIN ELECTROSTIMULATION

(71) Applicant: AGNES MEDICAL CO., LTD, Seongnam-si (KR)

(72) Inventors: Gunyoung AHN, Seongnam-si (KR); Hyuck Ki Hong, Yongin-si (KR); Kyoung Su Jung, Seongnam-si (KR); Moon Ki Shin, Seongnam-si (KR); Byoung Jun Cha, Namyangju-si (KR)

(73) Assignee: AGNES MEDICAL CO., LTD, Seongnam-si (KR)

(21) Appl. No.: 19/082,203

(22) Filed: Mar. 18, 2025

(30) Foreign Application Priority Data
Jul. 15, 2024 (KR) 10-2024-0093082

(51) Int. Cl. A61N 1/04

(52) U.S. Cl. CPC A61N 1/0472

(57) ABSTRACT

An electrode structure for skin electrostimulation includes an electrode plate made of a conductive material, and a plurality of prongs formed integrally with and made of the same material as the electrode plate, the plurality of prongs extending from one end of the electrode plate in the same direction as each other. End portions of the plurality of prongs are configured to be inserted into human skin to transmit electrical energy to the human skin.

### CROSS-REFERENCE TO PRIOR APPLICATION

This application claims priority to Korean Patent Application No. 10-2024-0093082 (filed on Jul. 15, 2024), which is all hereby incorporated by reference in its entirety.

### BACKGROUND

The present invention relates to an electrode structure for skin electrostimulation that is used in a skin treatment procedure, and more particularly, to an electrode structure for skin electrostimulation capable of removing a potential difference and a phase difference that may occur between a plurality of pins inserted into the skin and minimizing a depth error or an interval error that may occur due to assembly of the plurality of pins.

Much attention is being paid to a skin treatment method of maintaining skin elasticity and minimizing skin aging by directly transmitting electrical energy such as high-frequency current to the dermal layer of the skin through a pin or a needle to activate cell tissues.

Conventional skin treatment technology is disclosed in Related Art Patent Document KR 10-2013-0012805 A. Specifically, the above patent document discloses an electrode module including a plurality of needles and a base to which the needles are mounted. The plurality of needles is assembled to the base so as to be in contact with electric circuit patterns provided at the base and receives electrical energy supplied to the base. That is, the conventional electrode module has a structure in which the needles and the base configured to supply electrical energy to the needles are provided separately from each other, and the needles are assembled so as to be in contact with the circuit patterns of the base and thus are utilized as electrodes supplying electrical energy to the skin.

However, this conventional electrode structure has the following problems. Because the plurality of needles is aligned with each other and is assembled so as to be in contact with the circuit patterns of the base, a process of manufacturing the conventional electrode structure may be complicated and difficult. In addition, because the lengths of the circuit patterns in contact with the respective needles are not uniform, impedances before electrical energies reach the tips of the needles may not be uniform, and thus a potential difference may occur between the needles, leading to the occurrence of a difference between the supplied electrical energies.

In addition, due to an assembly error that inevitably occurs in the process of assembling the needles and the base, it may be difficult to form the plurality of needles so that the tip portions thereof have a uniform height. In addition, an error may occur in the arrangement angle between the plurality of needles, and the coupling strengths between the needles and the base may differ from each other. Therefore, when the needles penetrate the skin, there may be a difference in penetration depth between the needles.

### SUMMARY

The present invention has been made to solve the above problems, and it is an object of the present invention to provide an electrode structure for skin electrostimulation capable of removing a potential difference and a phase difference that may occur between a plurality of pins inserted into the skin and minimizing a depth error or an interval error that may occur due to assembly of the plurality of pins.

In addition, it is another object of the present invention to provide an electrode structure for skin electrostimulation including electrodes having a shape capable of forming a non-radiative high electric field and inducing heat diffusion in portions of pins inserted into the skin even when relatively low energy is applied to the pins inserted into the skin, thereby increasing treatment efficiency.

The objects of the present invention are not limited to the above-mentioned object, and other objects not mentioned herein will be clearly understood by those skilled in the art from the following description.

In accordance with the present invention, the above and other objects can be accomplished by the provision of an electrode structure for skin electrostimulation, the electrode structure including an electrode plate made of a conductive material and a plurality of prongs formed integrally with and made of the same material as the electrode plate, the plurality of prongs extending from one end of the electrode plate in the same direction as each other, wherein end portions of the plurality of prongs are inserted into a human skin to transmit electrical energy to the human skin.

In an embodiment of the present invention, the electrode plate may include an area to which an electrical signal for skin stimulation is applied from the outside. When the electrical signal is applied to the area, the electrode plate and the plurality of prongs may have the same electric potential as each other.

In an embodiment of the present invention, the electrode plate may include a plurality of protruding portions formed on an end thereof opposite the end thereof from which the plurality of prongs extends, and the plurality of protruding portions may be inserted into through-holes formed in a separate conductive plate.

In an embodiment of the present invention, the plurality of protruding portions and the end of the electrode plate on which the plurality of protruding portions is formed may serve as an area to which an electrical signal for skin stimulation is applied from the outside.

In an embodiment of the present invention, the plurality of prongs may include at least one prong having a first length allowing the tip thereof to reach a papillary dermis layer of the skin when penetrating the skin and at least one prong having a second length allowing the tip thereof to reach a reticular dermis layer of the skin when penetrating the skin.

In an embodiment of the present invention, each of the plurality of prongs comprises a tip portion formed to be gradually reduced in outer diameter toward a tip facing the skin and a body portion extending outward from a rear end of the tip portion and having a constant outer diameter, wherein the tip portion comprises a first tip edge portion formed by a pair of first tip inclined surfaces, a second tip edge portion formed by a pair of second tip inclined surfaces, and a pair of tip connection surfaces, each interconnecting one of the pair of first tip inclined surfaces and one of the pair of second tip inclined surfaces adjacent to each other, wherein the body portion comprises a first body edge portion formed by a pair of first body inclined surfaces, a second body edge portion formed by a pair of second body inclined surfaces, and a pair of body connection surfaces, each interconnecting one of the pair of first body inclined surfaces and one of the pair of second body inclined surfaces adjacent to each other, and wherein, when power is applied to the microblade, an electric field is emitted in a direction perpendicular to planar field emission surfaces formed on outer peripheries of the tip portion and the body portion and is transmitted to the skin.

In an embodiment of the present invention, each of the pair of tip connection surfaces is formed in a shape of an arc curved surface.

In an embodiment of the present invention, the tip portion comprises a pair of extension surfaces extending from the pair of body connection surfaces in a boundary area between the tip portion and the body portion so as to be connected to the pair of tip connection surfaces.

In an embodiment of the present invention, each of the pair of extension surfaces is formed in a shape of a planar surface.

In an embodiment of the present invention, an angle between the pair of first tip inclined surfaces and an angle between the pair of second tip inclined surfaces are acute angles.

In an embodiment of the present invention, an angle between each of the pair of first tip inclined surfaces and an adjacent one of the pair of tip connection surfaces and an angle between each of the pair of second tip inclined surfaces and an adjacent one of the pair of tip connection surfaces are obtuse angles.

In an embodiment of the present invention, a distance between a vertex of the first tip edge portion at which the pair of first tip inclined surfaces meet each other and a vertex of the second tip edge portion at which the pair of second tip inclined surfaces meet each other is longer than a distance between the pair of tip connection surfaces facing each other.

In an embodiment of the present invention, each of the pair of body connection surfaces is formed in a shape of a planar surface.

In an embodiment of the present invention, an angle between the pair of first body inclined surfaces and an angle between the pair of second body inclined surfaces are acute angles.

In an embodiment of the present invention, an angle between each of the pair of first body inclined surfaces and an adjacent one of the pair of body connection surfaces and an angle between each of the pair of second body inclined surfaces and an adjacent one of the pair of body connection surfaces are obtuse angles.

In an embodiment of the present invention, a distance between a vertex of the first body edge portion at which the pair of first body inclined surfaces meet each other and a vertex of the second body edge portion at which the pair of second body inclined surfaces meet each other is longer than a distance between the pair of body connection surfaces facing each other.

In an embodiment of the present invention, an end of the tip portion has a curved shape.

In an embodiment of the present invention, the tip portion and the body portion have a polyhedral shape with angled surfaces.

In an embodiment of the present invention, a portion of the tip portion and the body portion have a polygonal shape in a longitudinal section.

### BRIEF DESCRIPTION OF THE DRAWINGS

The above and other objects, features, and other advantages of the present invention will be more clearly understood from the following detailed description taken in conjunction with the accompanying drawings, in which:

FIGS. 1 and 2 are plan views of electrode structures for skin electrostimulation according to various embodiments of the present invention;

FIG. 3 is a perspective view schematically showing a module structure to which a plurality of electrode structures for skin electrostimulation according to an embodiment of the present invention is applied;

FIG. 4 is a side cross-sectional view schematically showing the module structure to which a plurality of electrode structures for skin electrostimulation according to an embodiment of the present invention is applied;

FIG. 5 is a perspective view showing the external appearance of a prong of electrode structures for skin electrostimulation according to various embodiments of the present invention;

FIG. 6A is a side view of the prong of electrode structures for skin electrostimulation according to various embodiments of the present invention;

FIG. 6B is a plan view of the prong of electrode structures for skin electrostimulation according to various embodiments of the present invention;

FIG. 7 is a view showing the cross-sectional shapes of a tip portion and a body portion of the prong of electrode structures for skin electrostimulation according to various embodiments of the present invention that vary in the longitudinal direction thereof;

FIG. 8A shows the distribution of an electric field emitted from the conventional conical or cylindrical pin;

FIG. 8B shows the distribution of an electric field emitted from the prong of electrode structures for skin electrostimulation according to various embodiments of the present invention;

FIG. 9A is a graph showing the electric field intensity at each position in the longitudinal direction of the conventional conical or cylindrical pin;

FIG. 9B is a graph showing the electric field intensity at each position in the longitudinal direction of the prong of electrode structures for skin electrostimulation according to various embodiments of the present invention;

FIG. 10A is a schematic cross-sectional diagram showing the electric field generation state in the longitudinal direction of the conventional conical or cylindrical pin;

FIG. 10B is a schematic cross-sectional diagram showing the electric field generation state in the longitudinal direction of the prong of electrode structures for skin electrostimulation according to various embodiments of the present invention;

FIG. 11A is a graph showing the electric field intensity in the conventional conical or cylindrical pin; and

FIG. 11B is a graph showing the electric field intensity in the prong of electrode structures for skin electrostimulation according to various embodiments of the present invention.

### DETAILED DESCRIPTION

Hereinafter, an electrode structure for skin electrostimulation according to an embodiment of the present invention will be described with reference to the drawings.

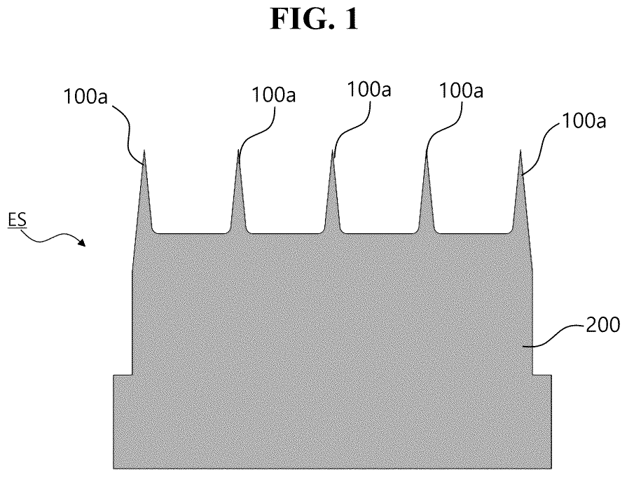

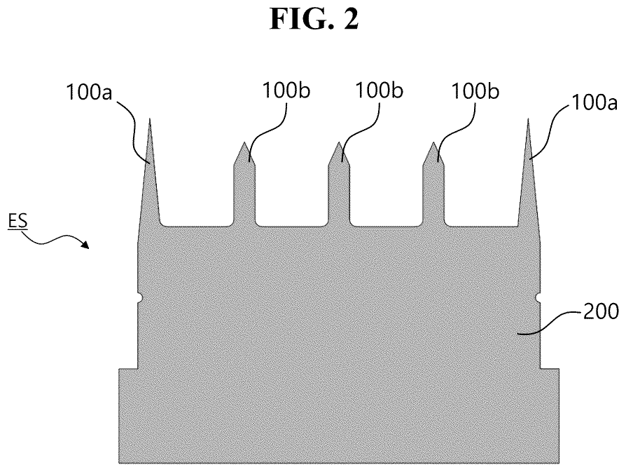

FIGS. 1 and 2 are plan views of electrode structures for skin electrostimulation according to various embodiments of the present invention.

Referring to FIGS. 1 and 2, an electrode structure ES for skin electrostimulation according to an embodiment of the present invention may include an electrode plate 200 made of a conductive material and a plurality of prongs 100a and 100b formed integrally with and made of the same material as the electrode plate 200 and extending from one end of the electrode plate 200 in the same direction as each other.

The electrode plate 200 may be made of a conductive material allowing current to flow therethrough, for example, a single metal or an alloy containing a plurality of metals. Although the electrode plate 200 is illustrated in FIG. 1 as having a substantially rectangular shape, the electrode plate 200 may be formed to have various other shapes as needed under various conditions, such as the environment in which the electrode plate 200 is used or the shapes of neighboring structures. Alternatively, the electrode plate 200 may be bent or curved such that both sides thereof, from which the prongs 100a and 100b do not extend, are bonded to each other, and thus may have a three-dimensional structure having a rectangular or circular cross-section.

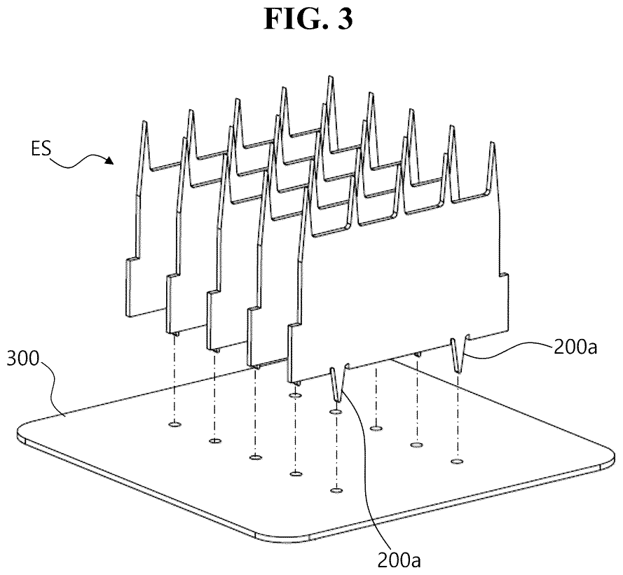

The electrode plate 200 may have an area, which is disposed in a handpiece or the like when assembled to a skin stimulation system and to which an electrical signal for skin stimulation is applied. The area to which the electrical signal is applied (hereinafter referred to as the “electrical-signal-receiving area”) may be variously defined depending on the assembly form of the electrode structure ES. For example, when a plurality of electrode structures ES is assembled so as to be disposed parallel to each other as shown in FIG. 3, the other end of the electrode plate 200, opposite the end thereof from which the prongs 100a and 100b extend, or the left and right sides thereof based on the illustrated direction may be defined as the electrical-signal-receiving area.

The prongs 100a and 100b may be implemented in the form of a pin or a blade extending from the electrode plate 200. The prongs 100a and 100b may be made of the same material as the electrode plate 200 and may be formed integrally with the electrode plate 200. That is, in the manufacturing process, the prongs 100a and 100b and the electrode plate 200 may be manufactured at the same time through a single process.

The prongs 100a and 100b may be exposed to the outside of the handpiece or the like when assembled to the skin stimulation system and may penetrate the skin. In the embodiment shown in FIG. 1, the prongs 100a may be formed such that the lengths by which the prongs 100a extend from the electrode plate 200 are identical to each other. In another example, as shown in FIG. 2, the prongs 100a and 100b may be formed such that the lengths by which the prongs 100a and 100b extend from the electrode plate 200 are different from each other. In the embodiment shown in FIG. 2, the prongs 100a and 100b extending from the electrode plate 200 may have two different lengths. In detail, the prongs 100b having a relatively short length may be formed so that the tips thereof reach the papillary dermis layer when penetrating the skin, and the prongs 100a having a relatively long length may be formed so that the tips thereof reach the reticular dermis layer when penetrating the skin.

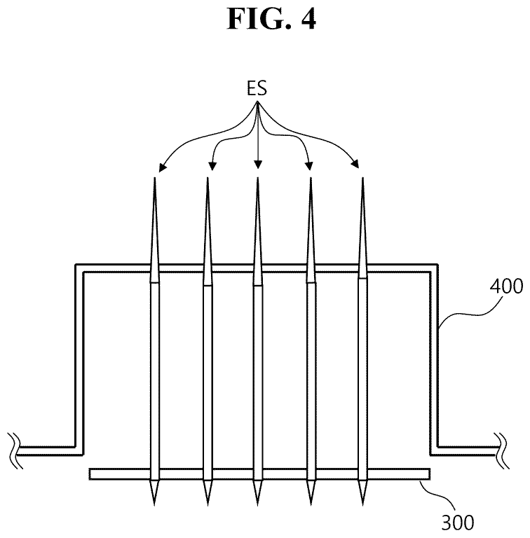

FIG. 3 is a perspective view schematically showing a module structure to which a plurality of electrode structures for skin electrostimulation according to an embodiment of the present invention is applied, and FIG. 4 is a side cross-sectional view schematically showing the module structure to which a plurality of electrode structures for skin electrostimulation according to an embodiment of the present invention is applied.

Referring to FIGS. 3 and 4, the electrode structure ES for skin electrostimulation according to an embodiment of the present invention may be disposed in a housing 400 configured to expose at least a portion of each of the prongs 100a and 100b to the outside and to accommodate the electrode plate 200 therein. A plurality of electrode structures ES may be disposed parallel to each other at regular intervals in order to form a two-dimensional pin arrangement structure on a plane. The tip and at least a portion of each prong 100 may be exposed to the outside of the housing 400 to penetrate into the skin tissue. When an electrical signal is applied to the electrical-signal-receiving area of the electrode plate 200, the prongs 100 may have the same electric potential as the electrode plate 200, and thus may act as electrodes capable of providing electric stimulation to the skin tissue.

In particular, the electrode structure ES for skin electrostimulation according to the embodiment of the present invention may be provided in plural, and the plurality of electrode structures ES for skin electrostimulation may be disposed on a conductive plate 300, which is disposed under the electrode plate 200 when assembled to the handpiece or the like of the skin stimulation system and has a surface perpendicular to the direction in which the prongs 100a and 100b extend. That is, the ends of the electrode plates 200, on which the prongs 100a and 100b are not formed, and the upper surface of the conductive plate 300 may be in contact with each other, whereby the electrode structures ES for skin electrostimulation may be supported on the conductive plate 300. In this case, the conductive plate 300 may have through-holes formed in portions thereof corresponding to the placement positions of the electrode structures ES for skin electrostimulation, and the electrode plates 200 may have a plurality of protruding portions 200a extending toward the conductive plate 300 from the ends thereof that are in contact with the conductive plate 300. The protruding portions 200a may be inserted into the through-holes formed in the conductive plate 300, whereby the electrode structures ES for skin electrostimulation may be aligned in a preset arrangement structure.

The conductive plate 300 may be made of a conductive material (e.g., metal), and may serve as a terminal that receives an electrical signal from the outside and transmits electrical signal to the electrode structures ES for skin electrostimulation.

In the example shown in FIGS. 3 and 4, the electrode plates 200 may have protruding portions 200a extending from the ends thereof, opposite the ends thereof from which the prongs 100a and 100b extend, in a direction opposite the direction in which the prongs 100a and 100b extend. Since the protruding portions 200a are inserted into the through-holes formed in the conductive plate 300 and thus the electrode structures ES for skin electrostimulation are aligned on the conductive plate 300, the ends of the electrode plates 200, opposite the ends thereof from which the prongs 100a and 100b extend, and the protruding portions 200a may be defined as the electrical-signal-receiving area through which an electrical signal is applied to the electrode structures ES for skin electrostimulation.

In addition, since the ends of the electrode plates 200 of the plurality of electrode structures ES for skin electrostimulation are supported together by the conductive plate 300 having an integral structure, the lengths by which all of the prongs 100a and 100b are exposed to the outside of the housing 400 (the lengths corresponding to depths to which all of the prongs are inserted into the skin) may be substantially identical to each other, and all of the prongs 100a and 100b may receive substantially the same repulsive force when inserted into the skin.

In particular, in the embodiment of the present invention, the prongs 100 may be formed in an anisotropic blade structure.

The concrete shape and structure of the prongs 100 will be described in more detail later.

The electrode structure for skin electrostimulation according to the embodiment of the present invention may have the following excellent advantages.

First, the electrode structure for skin electrostimulation according to the embodiment of the present invention has advantages in that the manufacturing process thereof is simple, and it is possible to easily and conveniently change the lengths of the prongs, the intervals therebetween, and the arrangement thereof.

The related art requires a process of manufacturing a plurality of individual needles (pins) and a process of bonding the manufactured needles to a separate base having circuit patterns. Therefore, in the related art, when the lengths of the plurality of needles, the intervals therebetween, and the arrangement thereof are to be changed, there is inconvenience of having to design and manufacture new needles, a new base having another size, and new circuit patterns in the base and then reassemble the newly manufactured components.

In contrast, the electrode structure for skin electrostimulation according to the embodiment of the present invention has an advantage of enabling immediate manufacture of prongs after change in design of the lengths of the prongs, the intervals therebetween, and the arrangement thereof and of not requiring any separate assembly process.

Next, the electrode structure for skin electrostimulation according to the embodiment of the present invention has an advantage of easily making the phases and the electric potentials of the plurality of prongs constant.

In the related art, it is difficult to make respective impedances from a generator generating an electrical signal to ends of the individual needles completely identical. For example, because the lengths of the circuit patterns in contact with the individual needles are different from each other, an error occurs in the electric potentials of the needles generated by electrical signals applied to the individual needles and the phases of the electrical signals.

In contrast, in the electrode structure for skin electrostimulation according to the embodiment of the present invention, since the plurality of prongs and the electrode plate are made of the same conductive material (metal) and are formed integrally with each other, the electrical signals applied to the plurality of prongs may have the same phase, and the plurality of prongs may have the same electric potential at a certain time point.

Next, the electrode structure for skin electrostimulation according to the embodiment of the present invention has an advantage of easily making the distances between the prongs (electrodes) constant.

A potential difference is required for generation of current, and current flows between a pair of electrodes due to the potential difference therebetween. In the related art, when it is intended to make the depths to which the ends of the plurality of pins are inserted into the skin completely constant, the precision may be affected by various factors, such as assembly tolerances between the individual pins and the base, an error in the assembly angle, and robustness of connection parts. That is, in the related art, an error may occur in the depths to which the ends of the respective pins are inserted into the skin due to various factors, such as bonding robustness, bonding tolerances, and bonding angles between the individual pins and the circuit board.

In contrast, in the electrode structure for skin electrostimulation according to the embodiment of the present invention, because the plurality of prongs is formed integrally with a single electrode plate, the lengths by which the prongs are exposed to the outside of the housing 400 are not affected by any factors other than manufacturing tolerances, and thus it is possible to minimize a difference between the depths to which the ends of the plurality of prongs are inserted into the skin.

In addition, the electrode structure for skin electrostimulation according to the embodiment of the present invention is advantageous in ensuring uniform insertion of the plurality of prongs into the skin.

In the related art, when the plurality of pins is simultaneously inserted into the skin tissue, the individual pins receive repulsive force in a direction opposite the insertion direction thereof. In this case, the ability of the bonded portions between the individual pins and the base to withstand the repulsive force may be a factor determining the uniformity of insertion. The uniformity of insertion may be varied depending on the elastic modulus of the individual bonded portions in the insertion direction. The bonded portions may be damaged when the repulsive force exceeds the shear force thereof.

In contrast, in the electrode structure for skin electrostimulation according to the embodiment of the present invention, because the plurality of prongs is formed integrally with a single electrode plate, repulsive force exerted on the respective prongs may be dispersed over the entire area of the single electrode plate, and accordingly, the uniformity of insertion of the plurality of prongs may be easily ensured.

In addition, the electrode structure for skin electrostimulation according to the embodiment of the present invention may be even more advantageous when employing blade-shaped prongs to be described later.

The blade-shaped prongs to be described later have an anisotropic structure on the vertical plane in the insertion direction into the skin. Accordingly, the electrical influence of the prongs on the skin tissue into which the prongs have been inserted is also anisotropic on the vertical plane. When multiple anisotropic electrostimulations are simultaneously applied to the skin tissue on the vertical plane through the plurality of prongs, it may also be important that the rotational angles of the respective prongs in the insertion direction thereof be formed to be uniform. For example, because aged skin undergoes anisotropic sagging, it is necessary to implement anisotropic skin tightening in order to treat saggy skin. In order to precisely implement skin tightening in a desired direction through electrostimulation to the skin tissue, the rotational angles of a plurality of electrodes need to be uniform.

The electrode structure for skin electrostimulation according to the embodiment of the present invention does not require an assembly process of individually bonding the plurality of prongs to the base. Therefore, the electrode structure for skin electrostimulation according to the embodiment of the present invention is advantageous in forming the rotational angles of the respective prongs in the insertion direction thereof to be uniform.

Hereinafter, the prongs of the electrode structure for skin electrostimulation according to the embodiment of the present invention will be described in more detail.

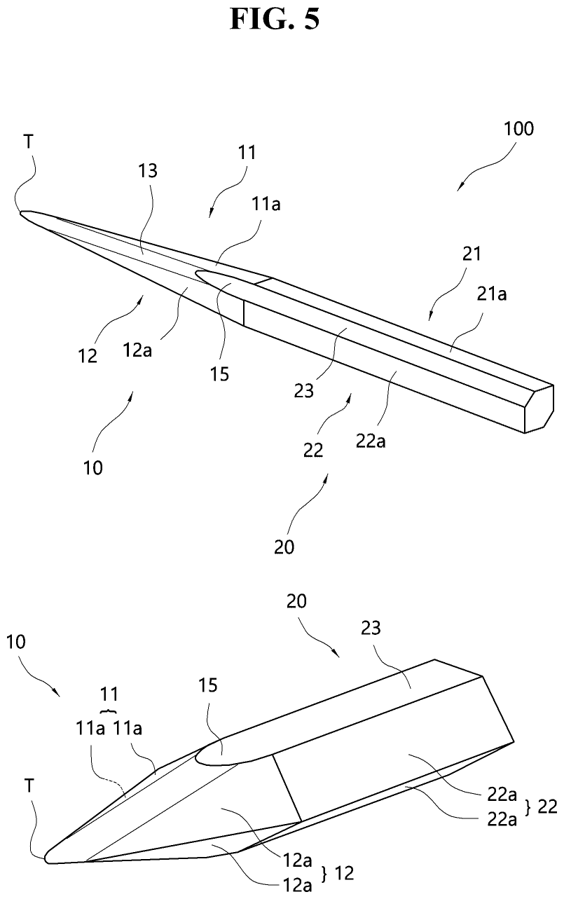

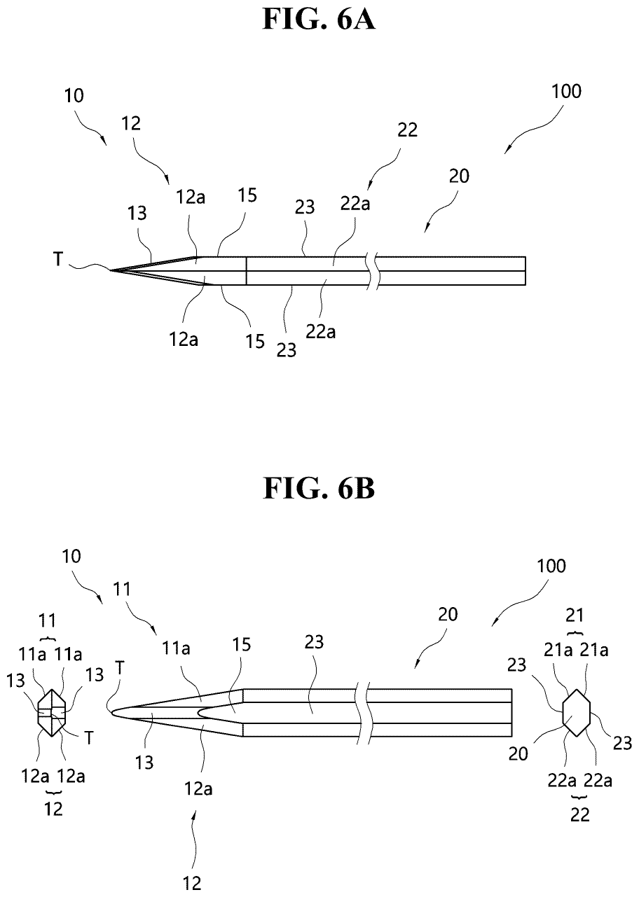

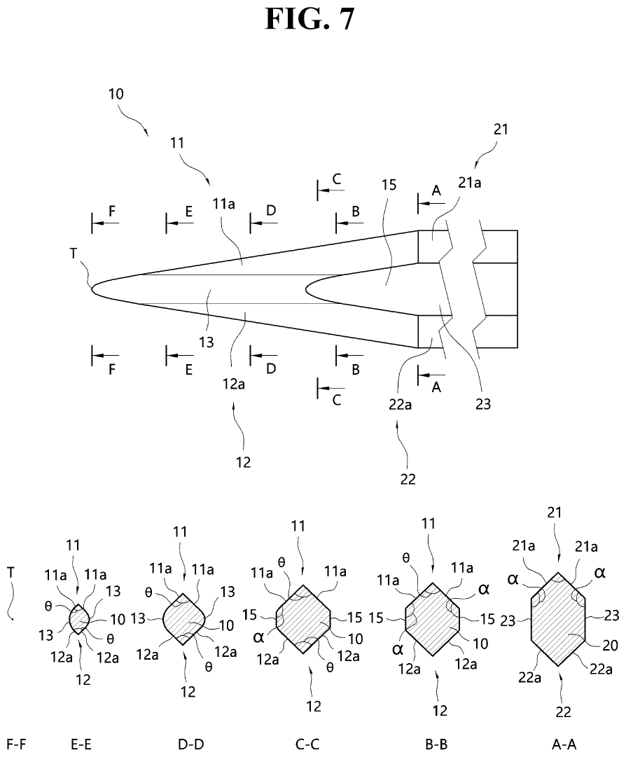

FIG. 5 is a perspective view showing the external appearance of a prong of electrode structures for skin electrostimulation according to various embodiments of the present invention. FIG. 6A is a side view of the prong of electrode structures for skin electrostimulation according to various embodiments of the present invention, and FIG. 6B is a plan view of the prong of electrode structures for skin electrostimulation according to various embodiments of the present invention. FIG. 7 is a view showing the cross-sectional shapes of a tip portion and a body portion of the prong of electrode structures for skin electrostimulation according to various embodiments of the present invention that vary in the longitudinal direction thereof.

As shown in FIGS. 5, 6A, 6B and 7, the prong 100 of electrode structures for skin electrostimulation according to various embodiments of the present invention may include a tip portion 10 and a body portion 20, which are made of a conductive material, are connected to an external power source, and are inserted to a predetermined depth in the skin to transmit electrical energy, which is an electric field generated when power is applied thereto, to the skin, thereby performing electrostimulation.

Referring to FIGS. 5, 6A, 6B, and 7, the tip portion 10 is a distal end portion that is gradually reduced in outer diameter toward a pointed tip T, which faces a site of the skin that will undergo a procedure.

The tip portion 10 may include a first tip edge portion 11 formed by a pair of first tip inclined surfaces 11a, a second tip edge portion 12 formed by a pair of second tip inclined surfaces 12a, and a pair of tip connection surfaces 13, each of which interconnects one of the pair of first tip inclined surfaces 11a and one of the pair of second tip inclined surfaces 12a adjacent to each other.

In this case, in accordance with the design shape of the tip portion 10 that is gradually reduced in outer diameter toward the pointed tip T, each of the pair of first tip inclined surfaces 11a and the pair of second tip inclined surfaces 12a may be formed in a shape of a planar surface that is gradually reduced in width toward the pointed tip T and is inclined at a predetermined angle toward the pointed tip T and the first and second tip edge portions.

In accordance with the design shape of the tip portion 10 that is gradually reduced in outer diameter toward the pointed tip T, each of the pair of tip connection surfaces 13 may be formed in a shape of a planar surface or a curved surface that is inclined at a predetermined angle toward the pointed tip T while maintaining a constant width and is connected to the pointed tip T.

In this case, each of the pair of tip connection surfaces 13 is preferably formed in a shape of a substantially arc curved surface in order to prevent damage to the skin when the pointed tip T of the tip portion 10 is brought into contact with and inserted into the skin.

Although each of the pair of tip connection surfaces 13 is illustrated and described as being formed in a shape of an arc curved surface, the invention is not limited thereto. Each of the pair of tip connection surfaces 13 may be formed in a shape of a planar surface in order to emit electrical energy, which is an electric field transmitted to the skin through the tip connection surfaces 13, in a direction perpendicular thereto. In addition, the thickness of the tip portion 10 decreases toward the pointed end T, and finally, the shape of the pointed end T may have a convex curved shape.

The tip portion 10 may include a pair of extension surfaces 15 extending a predetermined length from a pair of body connection surfaces 23 in a boundary area between the tip portion 10 and the body portion 20 so as to be connected to the pair of tip connection surfaces 13 inclined at a predetermined angle.

Here, each of the pair of extension surfaces 15 is preferably formed in a shape of a planar surface in order to emit electrical energy, which is an electric field transmitted to the skin when power is applied, in a direction perpendicular thereto together with the pair of body connection surfaces 23 formed in a shape of a planar surface.

An angle θ between the pair of first tip inclined surfaces 11a forming the first tip edge portion 11 and an angle θ between the pair of second tip inclined surfaces 12a forming the second tip edge portion 12 are preferably acute angles smaller than approximately 90° so that the tip portion is easily inserted into the skin by external force.

An angle α between each of the pair of first tip inclined surfaces 11a and an adjacent one of the pair of tip connection surfaces 13 and an angle α between each of the pair of second tip inclined surfaces 12a and an adjacent one of the pair of tip connection surfaces 13 are preferably obtuse angles larger than approximately 90°.

In addition, a vertical distance between the vertex of the first tip edge portion 11 at which the pair of first tip inclined surfaces 11a meet each other and the vertex of the second tip edge portion 12 at which the pair of second tip inclined surfaces 12a meet each other is preferably longer than a horizontal distance between the pair of tip connection surfaces 13 facing each other.

In this case, the vertical distance and the horizontal distance are shortened at the same rate in accordance with the shape design of the tip portion 10 that is gradually reduced in outer diameter toward the pointed tip T.

Accordingly, compared to the conventional conical or cylindrical pin having a substantially circular cross-section, the tip portion 10, which includes the pair of first tip inclined surfaces 11a, the pair of second tip inclined surfaces 12a, and the pair of tip connection surfaces 13, has a polygonal (e.g., substantially hexagonal) cross-section, and is provided on the outer periphery thereof with a plurality of field emission surfaces that emit electrical energy, which is an electric field emitted to the skin when power is applied, in a direction perpendicular thereto.

Due to the structural features as described above, the prong of electrode structures for skin electrostimulation according to various embodiments of the present invention has a polyhedral shape with angled surfaces as a whole, and may have a polygonal shape in a longitudinal section in a specific area. In addition, while the wound formed on the skin by the penetration of conventional conical or cylindrical pins has a shape of a dot, the wound formed by the prong of electrode structures for skin electrostimulation according to various embodiments of the present invention may be formed in a straight shape.

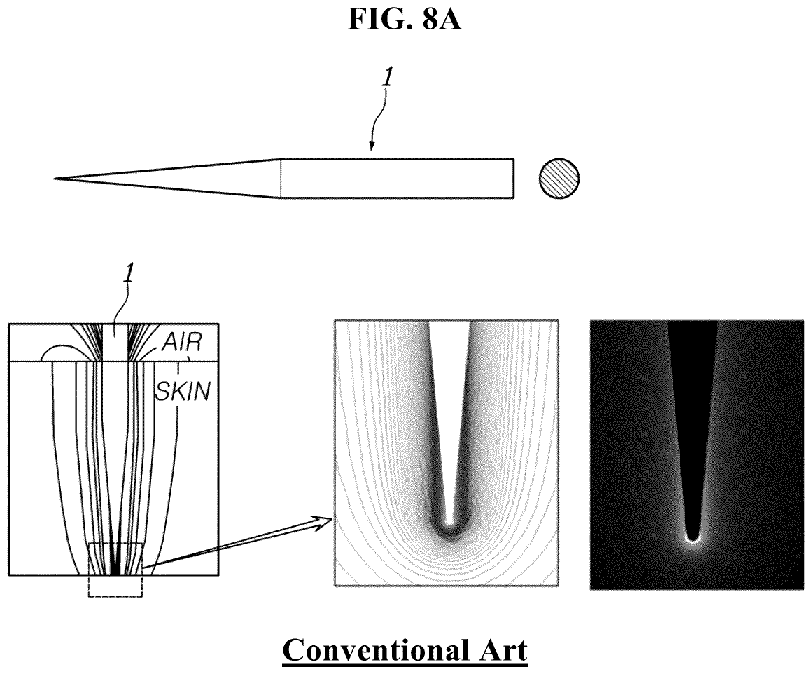

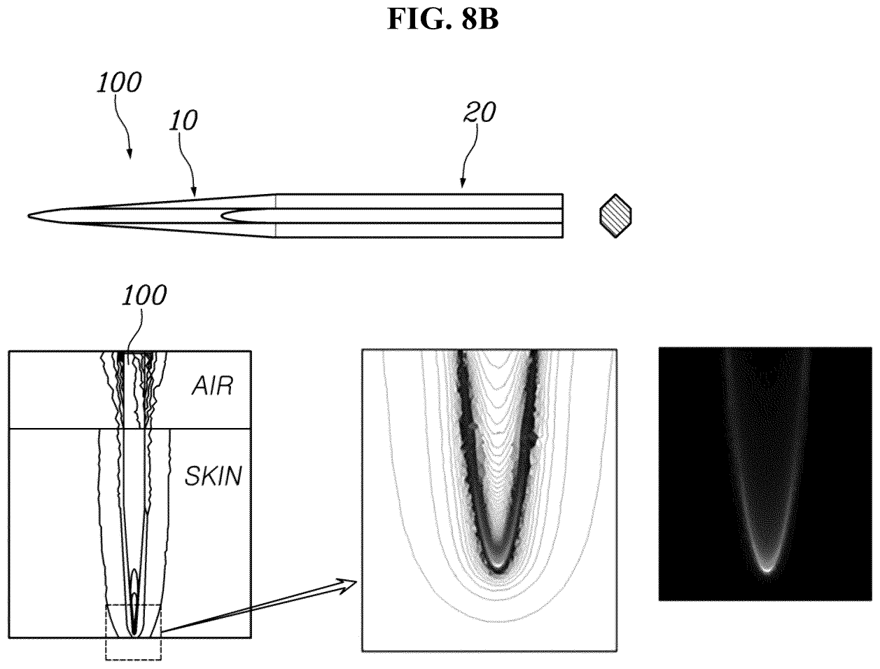

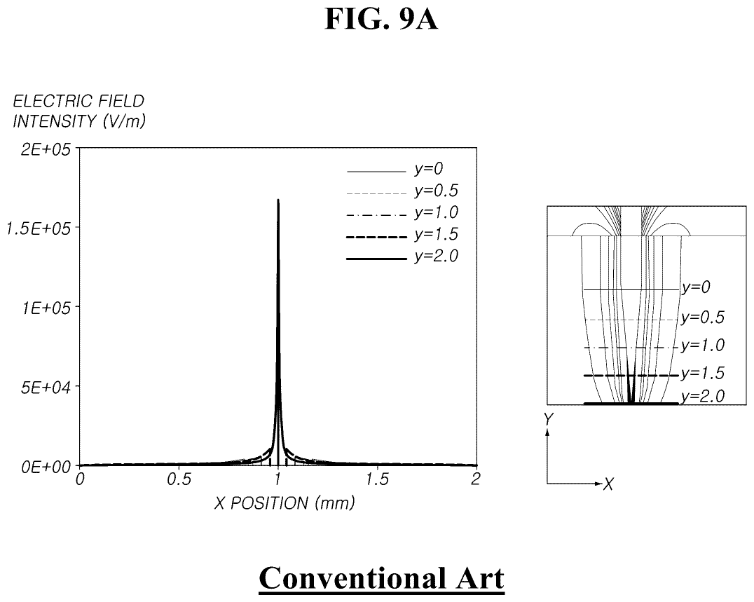

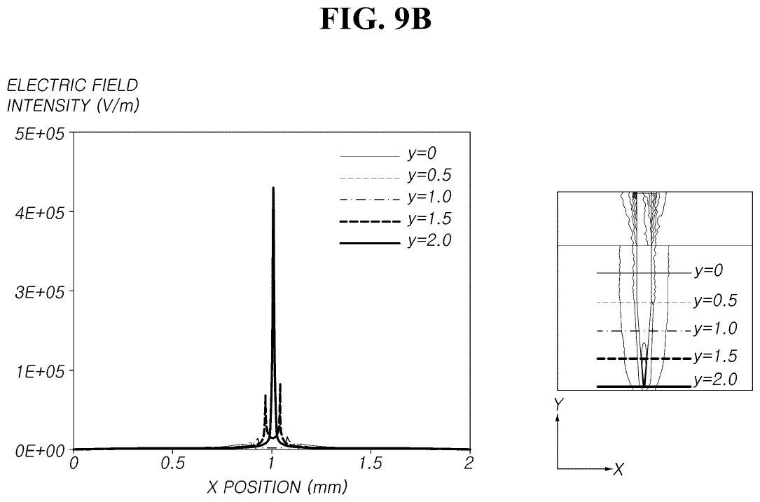

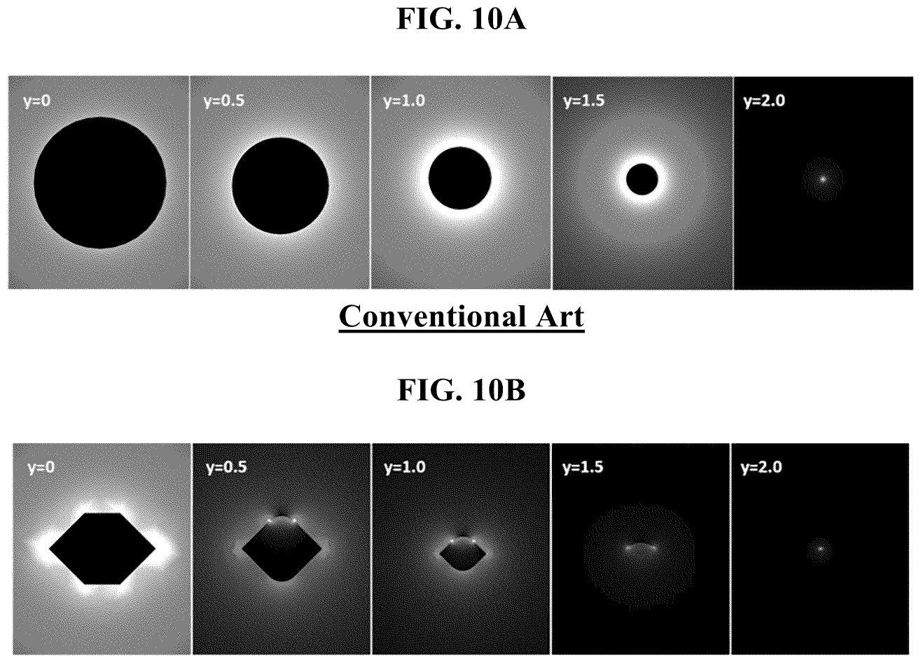

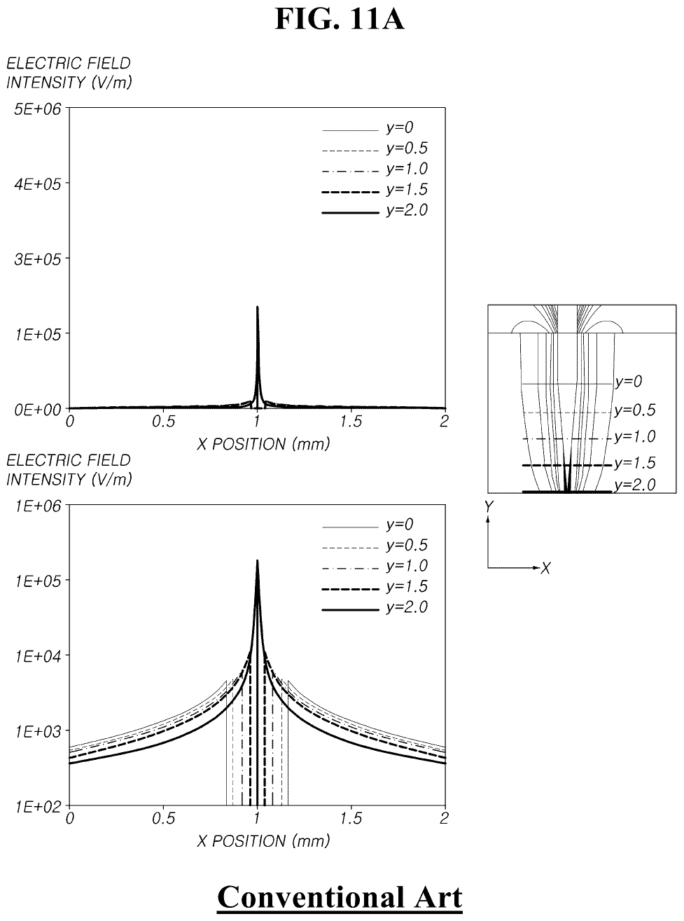

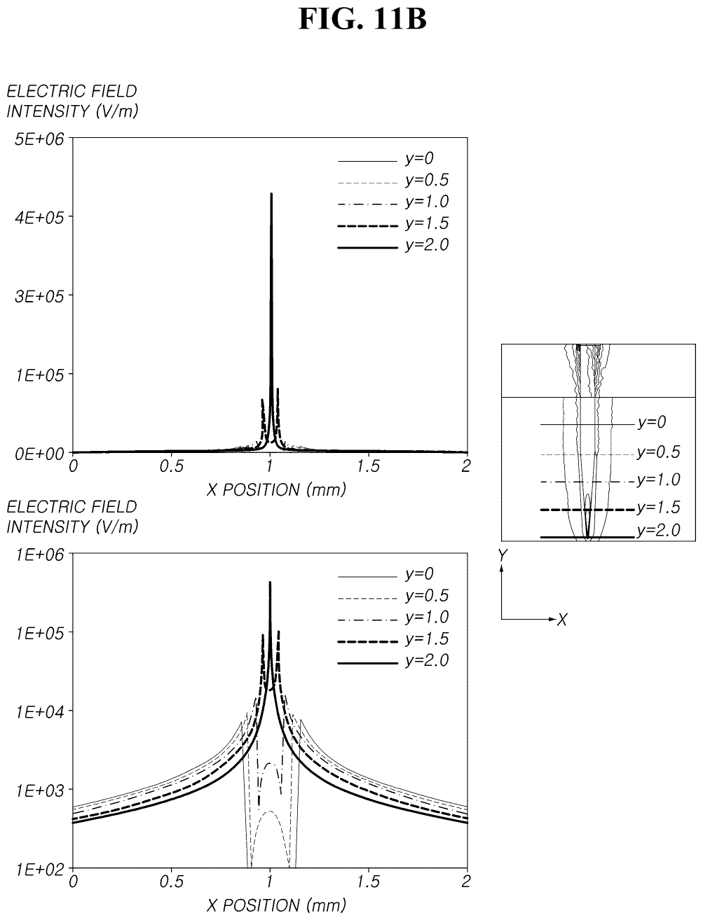

FIG. 8A shows the distribution of an electric field emitted from the conventional conical or cylindrical pin, and FIG. 8B shows the distribution of an electric field emitted from the prong of electrode structures for skin electrostimulation according to various embodiments of the present invention. FIG. 9A is a graph showing the electric field intensity at each position in the longitudinal direction of the conventional conical or cylindrical pin, and FIG. 9B is a graph showing the electric field intensity at each position in the longitudinal direction of the prong of electrode structures for skin the electrostimulation according to various embodiments oft present invention. FIG. 10A is a schematic cross-sectional diagram showing the electric field generation state in the longitudinal direction of the conventional conical or cylindrical pin, and FIG. 10B is a schematic cross-sectional diagram showing the electric field generation state in the longitudinal direction of the prong of electrode structures for skin electrostimulation according to various embodiments of the present invention. FIG. 11A is a graph showing the electric field intensity in the conventional conical or cylindrical pin, and FIG. 11B is a graph showing the electric field intensity in the prong of electrode structures for skin electrostimulation according to various embodiments of the present invention.

Referring to FIGS. 8A and 8B, it can be confirmed from simulation data about the electric field distribution that, when power is applied, the electric field is concentrated on the pointed tip of the conical or cylindrical pin 1 and the pointed tip of the tip portion of the prong 100 of electrode structures for skin electrostimulation according to various embodiments of the present invention and is emitted therefrom with high intensity.

Referring to FIGS. 8A, 9A, 10A, and 11A, in the case of the conical or cylindrical pin 1 having a circular cross-section, the electric field that is emitted to the skin is distributed non-uniformly with respect to the longitudinal direction thereof and has low intensity. In contrast, referring to FIGS. 8B, 9B, 10B, and 11B, in the case of the tip portion 10 of the prong 100 of electrode structures for skin electrostimulation according to various embodiments of the present invention, which includes a plurality of planar field emission surfaces, i.e., the pair of first tip inclined surfaces 11a, the pair of second tip inclined surfaces 12a, and the pair of tip connection surfaces 13, the electric field that is emitted to the skin is distributed uniformly with respect to the longitudinal direction thereof and has relatively high intensity.

In particular, referring to FIGS. 9A and 9B, it can be seen that the intensity of the electric field emitted to the skin from the tip portion 10, which includes the pair of first tip inclined surfaces 11a, the pair of second tip inclined surfaces 12a, and the pair of tip connection surfaces 13, is greater than the intensity of the electric field emitted and transmitted to the skin from the conventional conical or cylindrical pin 1.

Referring to FIGS. 10A and 10B, it can be seen that electrical energy, which is an electric field emitted from the pair of first tip inclined surfaces 11a, the pair of second tip inclined surfaces 12a, and the pair of tip connection surfaces 13 of the tip portion, is more strongly emitted and transmitted to the skin in a direction perpendicular to the planar surfaces.

Accordingly, when power is applied to the prong 100 in the state in which the tip portion is inserted to a predetermined depth in the skin together with the body portion, electrical energy, which is an electric field, is transmitted to the skin with high intensity from the planar field emission surfaces, i.e., the pair of first tip inclined surfaces 11a, the pair of second tip inclined surfaces 12a, and the pair of tip connection surfaces 13, in a direction substantially perpendicular to the planar field emission surfaces. As a result, the efficiency of a skin treatment procedure using electrostimulation may be improved compared to the conventional conical or cylindrical pin.

In addition, compared to the conventional pin 1 having a substantially circular cross-section, the contact area between the tip portion 10, which includes the pair of first tip inclined surfaces 11a, the pair of second tip inclined surfaces 12a, and the pair of tip connection surfaces 13 and thus has a polygonal cross-section, and the skin is increased, whereby electrical energy, which is an electric field generated upon application of power, may be more efficiently emitted and transmitted to the skin.

Referring to FIGS. 5, 6A, 6B, and 7, the body portion 20 is a bar member that extends a predetermined length outwardly from the rear end of the tip portion 10, which is first inserted into the skin. The body portion 20 has a constant outer diameter so as to be inserted into the skin together with the tip portion 10 and is electrically connected to an external power source. The body portion 20 may include a first body edge portion 21 formed by a pair of first body inclined surfaces 21a, a second body edge portion 22 formed by a pair of second body inclined surfaces 22a, and a pair of body connection surfaces 23, each of which interconnects one of the pair of first body inclined surfaces 21a and one of the pair of second body inclined surfaces 22a adjacent to each other.

In this case, in accordance with the design shape of the body portion 20 that has a constant outer diameter in the longitudinal direction thereof, each of the pair of first body inclined surfaces 21a and the pair of second body inclined surfaces 22a may be formed in a shape of a planar surface that has a constant width and is inclined at a predetermined angle toward the vertices of the first and second body edge portions.

In accordance with the design shape of the body portion 20 that has a constant outer diameter in the longitudinal direction thereof, each of the pair of body connection surfaces 23 may be formed in a shape of a planar surface that has a constant width and is connected to a respective one of the pair of tip connection surfaces 13 of the tip portion via a respective one of the pair of extension surfaces 15.

Although each of the pair of body connection surfaces 23 is illustrated and described as being formed in a shape of a planar surface in order to emit electrical energy, which is an electric field transmitted to the skin when power is applied, in a direction perpendicular thereto, the invention is not limited thereto. Each of the pair of body connection surfaces 23 may be formed in a shape of an arc curved surface.

An angle θ between the pair of first body inclined surfaces 21a forming the first body edge portion 21 and an angle θ between the pair of second body inclined surfaces 22a forming the second body edge portion 22 are preferably acute angles smaller than approximately 90°.

An angle α between each of the pair of first body inclined surfaces 21a and an adjacent one of the pair of body connection surfaces 23 and an angle α between each of the pair of second body inclined surfaces 22a and an adjacent one of the pair of body connection surfaces 23 are preferably obtuse angles larger than approximately 90°.

In addition, a vertical distance between the vertex of the first body edge portion 21 at which the pair of first body inclined surfaces 21a meet each other and the vertex of the second body edge portion 22 at which the pair of second body inclined surfaces 22a meet each other is preferably longer than a horizontal distance between the pair of body connection surfaces 23 facing each other.

In this case, the vertical distance and the horizontal distance are maintained constant in accordance with the shape design of the body portion 20 that has a constant outer diameter in the longitudinal direction thereof.

Accordingly, compared to the conventional conical or cylindrical pin having a substantially circular cross-section, the body portion 20, which includes the pair of first body inclined surfaces 21a, the pair of second body inclined surfaces 22a, and the pair of body connection surfaces 23, has a polygonal (e.g., substantially hexagonal) cross-section, similar to the tip portion, and is provided on the outer periphery thereof with a plurality of field emission surfaces that emit electrical energy, which is an electric field emitted to the skin when power is applied, in a direction perpendicular thereto.

Referring to FIGS. 8A, 9A, 10A, and 11A, in the case of the conical or cylindrical pin 1 having a circular cross-section, the electric field that is emitted to the skin is distributed non-uniformly with respect to the longitudinal direction thereof and has low intensity. In contrast, referring to FIGS. 8B, 9B, 10B, and 11B, in the case of the body portion 100 of 20 of the prong electrode structures for skin electrostimulation according to various embodiments of the present invention, which includes a plurality of planar field emission surfaces, i.e., the pair of first body inclined surfaces 21a, the pair of second body inclined surfaces 22a, and the pair of body connection surfaces 23, similar to the tip portion 10, the electric field that is emitted to the skin is distributed uniformly with respect to the longitudinal direction thereof and has relatively high intensity.

In particular, referring to FIGS. 9A and 9B, it can be seen that the intensity of the electric field emitted to the skin from the body portion 20, which includes the pair of first body inclined surfaces 21a, the pair of second body inclined surfaces 22a, and the pair of body connection surfaces 23, is greater than the intensity of the electric field emitted and transmitted to the skin from the conventional conical or cylindrical pin 1.

Referring to FIGS. 10A and 10B, it can be seen that electrical energy, which is an electric field emitted from the pair of first body inclined surfaces 21a, the pair of second body inclined surfaces 22a, and the pair of body connection surfaces 23 of the body portion, is more strongly emitted and transmitted to the skin in a direction perpendicular to the planar surfaces.

Accordingly, when power is applied to the prong 100 in the state in which the body portion is inserted to a predetermined depth in the skin together with the tip portion, electrical energy, which is an electric field, is transmitted to the skin with high intensity from the planar field emission surfaces, i.e., the pair of first body inclined surfaces 21a, the pair of second body inclined surfaces 22a, and the pair of tip connection surfaces 23, in a direction substantially perpendicular to the planar field emission surfaces. As a result, the efficiency of a skin treatment procedure using electrostimulation may be improved compared to the conventional conical or cylindrical pin.

In other words, the conventional conical or cylindrical pin 1 has a radial electric field formed around the pin, but the prong 100 of electrode structures for skin electrostimulation according to various embodiments of the present invention the embodiment of the present invention forms a non-radial electric field, thereby inducing non-radial heat diffusion. Also, the prong 10 according to the embodiment of the present invention may induce skin contraction having a directionality when the skin penetrates by forming a non-radial electric field.

In addition, compared to the conventional pin 1 having a substantially circular cross-section, the contact area between the body portion 20, which includes the pair of first body inclined surfaces 21a, the pair of second body inclined surfaces 22a, and the pair of body connection surfaces 23 and thus has a polygonal cross-section, and the skin is increased, whereby electrical energy, which is an electric field generated upon application of power, may be more efficiently emitted and transmitted to the skin.

The features as described above are unique features arising from the structure of the prong according to the embodiment of the present invention and are unique that cannot be implemented by conventional conical or cylindrical pins.

As is apparent from the above description, according to the electrode structure for skin electrostimulation according to the embodiment of the present invention configured as described above, since a plurality of prongs extends from a single electrode plate in an integral form, it is possible to easily manufacture the plurality of prongs so that the intervals therebetween are constant and there is no length difference therebetween.

In addition, according to the electrode structure for skin electrostimulation according to the embodiment of the present invention, since the electrode plate and the plurality of prongs are implemented as an integral conductive body, the plurality of prongs may have the same electric potential when penetrating the skin, and it is possible to prevent the occurrence of a difference in skin insertion depth or insertion angle between the plurality of prongs.

In addition, according to the electrode structure for skin electrostimulation according to the embodiment of the present invention, the blade-shaped prong designed to be inserted into the skin includes a tip portion and a main body portion, each of which includes a first edge portion formed by a pair of first inclined surfaces, a second edge portion formed by a pair of second inclined surfaces, and a pair of connection surfaces, each of which interconnects one of the pair of first inclined surfaces and one of the pair of second inclined surfaces adjacent to each other, and thus has a polygonal cross-section. That is, the electrode structure for skin electrostimulation according to the present invention includes a plurality of planar field emission surfaces that emit and transmit an electric field to the skin in a direction perpendicular thereto. Accordingly, compared to the conventional conical or cylindrical pin, an electric field having specific directivity is emitted to the skin with high intensity. As a result, the present invention may increase the efficiency of a skin treatment procedure with a given amount of supplied energy.

In detail, the conventional conical or cylindrical pin may emit constant radiative energy and thus may not induce directivity of skin tightening. In contrast, according to the electrode structure for skin electrostimulation according to the embodiment of the present invention, the blade-shaped prong may form a non-radiative electric field and thus may induce non-radiative heat diffusion. In addition, the blade-shaped prong may induce skin tightening having directivity when penetrating the skin by forming a non-radiative electric field.

In addition, according to the electrode structure for skin electrostimulation according to the embodiment of the present invention, since the prong, which is inserted into the skin, has a blade shape, the prong may obtain the same electric field effect from a smaller amount of supplied energy and may obtain approximately two-fold higher electric field effect from the same amount of supplied energy compared to the conventional conical or cylindrical pin. In other words, the blade-shaped prong of the present invention may generate an electric field having relatively high intensity from relatively low energy.

In addition, according to the electrode structure for skin electrostimulation according to the embodiment of the present invention, since the prong inserted into the skin emits a plurality of electric fields having different intensities and directivities through a plurality of planar field emission surfaces of the tip portion and the main body portion thereof, each having a polygonal cross-section, it is possible to increase skin treatment effects through interaction with another adjacent prong.

The effects of the present invention are not limited to the above-mentioned effects, and it should be understood that the effects include all effects that can be inferred from the configuration of the invention described in the detailed description or appended claims of the present invention.

Although specific embodiments of the present invention have been disclosed for illustrative purposes, those skilled in the art will appreciate that various modifications, additions, and substitutions are possible, without departing from the scope and spirit of the invention as disclosed in the accompanying claims.

What is claimed is:

1. An electrode structure for skin electrostimulation, the electrode structure comprising:
an electrode plate made of a conductive material; and
a plurality of prongs formed integrally with and made of the same material as the electrode plate, the plurality of prongs extending from one end of the electrode plate in the same direction as each other,
wherein end portions of the plurality of prongs are configured to be inserted into a human skin to transmit electrical energy to the human skin.

2. The electrode structure according to claim 1, wherein the electrode plate comprises an area to which an electrical signal for skin stimulation is applied from an outside, and
wherein, when the electrical signal is applied to the area, the electrode plate and the plurality of prongs have the same electric potential as each other.

3. The electrode structure according to claim 1, wherein the electrode plate comprises a plurality of protruding portions formed on an end thereof opposite the one end thereof from which the plurality of prongs extends, and
wherein the plurality of protruding portions is inserted into through-holes formed in a separate conductive plate.

4. The electrode structure according to claim 3, wherein the plurality of protruding portions and the end of the electrode plate on which the plurality of protruding portions is formed serve as an area to which an electrical signal for skin stimulation is applied from an outside.

5. The electrode structure according to claim 1, wherein the plurality of prongs comprises:
at least one prong having a first length allowing a tip thereof to reach a papillary dermis layer of the skin when penetrating the skin; and
at least one prong having a second length allowing a tip thereof to reach a reticular dermis layer of the skin when penetrating the skin.

6. The electrode structure according to claim 1, wherein each of the plurality of prongs comprises:
a tip portion formed to be gradually reduced in outer diameter toward a tip facing the skin; and
a body portion extending outward from a rear end of the tip portion and having a constant outer diameter,
wherein the tip portion comprises a first tip edge portion formed by a pair of first tip inclined surfaces, a second tip edge portion formed by a pair of second tip inclined surfaces, and a pair of tip connection surfaces, each interconnecting one of the pair of first tip inclined surfaces and one of the pair of second tip inclined surfaces adjacent to each other,
wherein the body portion comprises a first body edge portion formed by a pair of first body inclined surfaces, a second body edge portion formed by a pair of second body inclined surfaces, and a pair of body connection surfaces, each interconnecting one of the pair of first body inclined surfaces and one of the pair of second body inclined surfaces adjacent to each other, and
wherein, when power is applied to the microblade, an electric field is emitted in a direction perpendicular to planar field emission surfaces formed on outer peripheries of the tip portion and the body portion and is transmitted to the skin.

7. The electrode structure according to claim 6, wherein each of the pair of tip connection surfaces is formed in a shape of an arc curved surface.

8. The electrode structure according to claim 6, wherein the tip portion comprises a pair of extension surfaces extending from the pair of body connection surfaces in a boundary area between the tip portion and the body portion so as to be connected to the pair of tip connection surfaces.

9. The electrode structure according to claim 8, wherein each of the pair of extension surfaces is formed in a shape of a planar surface.

10. The electrode structure according to claim 6, wherein an angle between the pair of first tip inclined surfaces and an angle between the pair of second tip inclined surfaces are acute angles.

11. The electrode structure according to claim 6, wherein an angle between each of the pair of first tip inclined surfaces and an adjacent one of the pair of tip connection surfaces and an angle between each of the pair of second tip inclined surfaces and an adjacent one of the pair of tip connection surfaces are obtuse angles.

12. The electrode structure according to claim 6, wherein a distance between a vertex of the first tip edge portion at which the pair of first tip inclined surfaces meet each other and a vertex of the second tip edge portion at which the pair of second tip inclined surfaces meet each other is longer than a distance between the pair of tip connection surfaces facing each other.

13. The electrode structure according to claim 6, wherein each of the pair of body connection surfaces is formed in a shape of a planar surface.

14. The electrode structure according to claim 6, wherein an angle between the pair of first body inclined surfaces and an angle between the pair of second body inclined surfaces are acute angles.

15. The electrode structure according to claim 6, wherein an angle between each of the pair of first body inclined surfaces and an adjacent one of the pair of body connection surfaces and an angle between each of the pair of second body inclined surfaces and an adjacent one of the pair of body connection surfaces are obtuse angles.

16. The electrode structure according to claim 6, wherein a distance between a vertex of the first body edge portion at which the pair of first body inclined surfaces meet each other and a vertex of the second body edge portion at which the pair of second body inclined surfaces meet each other is longer than a distance between the pair of body connection surfaces facing each other.

17. The electrode structure according to claim 6, wherein an end of the tip portion has a curved shape.

18. The electrode structure according to claim 6, wherein the tip portion and the body portion have a polyhedral shape with angled surfaces.

19. The electrode structure according to claim 6, wherein a portion of the tip portion and the body portion have a polygonal shape in a longitudinal section.
